(19) United States
(12) Patent Application Publication
(10) Pub. No.: US 2026/0322172 A1
(43) Pub. Date: Oct. 15, 2026

(54) WIRELESS POWER TRANSMISSION DEVICE

(71) Applicant: Murata Manufacturing Co., Ltd., Kyoto-fu (JP)

(72) Inventors: Norikazu SAKAMOTO, Nagaokakyo-shi (JP); Tatsuya HOSOTANI, Nagaokakyo-shi (JP)

(73) Assignee: Murata Manufacturing Co., Ltd., Kyoto-fu (JP)

(21) Appl. No.: 19/690,821

(22) Filed: May 28, 2026

Related U.S. Application Data
(63) Continuation of application No. PCT/JP2024/037721, filed on Oct. 23, 2024.

(30) Foreign Application Priority Data
Dec. 22, 2023 (JP) 2023-216610

(51) Int. Cl. H02J 50/12; A61N 1/378; H01F 27/08; H02J 50/00; H02J 50/70

(52) U.S. Cl. CPC H02J 50/12; H01F 27/08; H02J 50/005; H02J 50/70; A61N 1/3787

(57) ABSTRACT

A wireless power transmission device includes an external housing including a first wall on a living body facing side and a second wall on a living body opposite side, a power transmission coil in the external housing, a power transmission resonant capacitor that constitutes a resonant circuit together with the power transmission coil, a magnetic sheet in the external housing, and a power transmission circuit board, in the external housing, on which a power semiconductor element handling a DC power source is mounted. The power transmission coil, the magnetic sheet, and the power transmission circuit board are disposed in this order from the first wall toward the second wall. In the power transmission circuit board, a mount surface of the power semiconductor element faces the magnetic sheet.

### CROSS-REFERENCE TO RELATED APPLICATIONS

This application claims benefit of priority to International Patent Application No. PCT/JP2024/037721, filed Oct. 23, 2024, and to Japanese Patent Application No. 2023-216610, filed Dec. 22, 2023, the entire contents of each are incorporated herein by reference.

### BACKGROUND

### Technical Field

The present disclosure relates to an implantable medical device, embedded in a living body, that wirelessly receives power from an outside of the living body.

### Background Art

In recent years, implantable medical devices concerning neurostimulation and neuro-sensing have been researched and developed. In such implantable medical devices, multifunctionality and multi-channeling are advancing.

As a result, in the implantable medical devices, there is a problem with increase in power consumption and burden on patients during replacement of the battery. Wireless power feeding needs to be implemented in such implantable medical devices to reduce this burden.

Japanese Unexamined Patent Application Publication No. 2010-258437 describes a system that wirelessly supplies power to a drive unit that is externally attached to both feet of a person, as an example of general wireless power feeding.

### SUMMARY

Such implantable medical devices have an increased power consumption due to multifunctionality and the like. Accordingly, a wireless power transmission device for implantable medical devices needs to achieve high power efficiency of a wireless power feeding system. In the wireless power transmission device for implantable medical devices, power loss caused by an increase in the transmission power becomes Joule heat and causes heat generation, a temperature rise tends to be large.

Accordingly, in the wireless power transmission device for implantable medical devices, it is necessary to perform the temperature control and the suppression of heat that affects the living body in which a medical device is implanted. In particular, a power transmission device for medical devices requires waterproof and dustproof specifications and a sealed structure. In a sealed structure, a temperature rise of a wireless power transmission device that incorporates a power transmission circuit and a power transmission coil that serve as heat sources becomes a greater issue, and accordingly, the stricter temperature control and the suppression of heat that affects the living body are required.

Therefore, the present disclosure suppresses the effect of heat generation on the living body and suppress a temperature rise of the living body while achieving high power efficiency in wireless power feeding in the wireless power transmission device for implantable medical devices.

A wireless power transmission device according to an embodiment of the present disclosure includes an external housing including a first wall on a living body facing side and a second wall on a living body opposite side, a power transmission coil disposed in the external housing, a power transmission resonant capacitor that constitutes a resonant circuit together with the power transmission coil, a magnetic sheet disposed in the external housing, and a power transmission circuit board, disposed in the external housing, on which a power semiconductor element handling a DC power source is mounted. The power transmission coil, the magnetic sheet, and the power transmission circuit board are disposed in this order from the first wall toward the second wall.

In the power transmission circuit board, a mount surface of the power semiconductor element faces the magnetic sheet.

The wireless power transmission device further includes a first TIM material, disposed between the power semiconductor element and the magnetic sheet, that is in contact with the power semiconductor element and the magnetic sheet, a second TIM material, disposed between the power transmission circuit board and the second wall, that is in contact with the power transmission circuit board and the second wall, and a heat insulation sheet that is disposed closer to the first wall than is the power transmission coil or a gap between the power transmission coil and the first wall.

The wireless power transmission device forms a first heat path that passes through the first TIM material and has a first average thermal resistance. The wireless power transmission device forms a second heat path that passes through the second TIM material and has a second average thermal resistance. The wireless power transmission device forms a third heat path that includes the heat insulation sheet or the gap and has a third average thermal resistance. The third average thermal resistance is higher than the first average thermal resistance and the second average thermal resistance.

A wireless power transmission device according to an embodiment of the present disclosure includes an external housing including a first wall on a living body facing side and a second wall on a living body opposite side, a power transmission coil disposed in the external housing, a power transmission resonant capacitor that constitutes a resonant circuit together with the power transmission coil, a magnetic sheet disposed in the external housing, and a power transmission circuit board, disposed in the external housing, on which a power semiconductor element handling a DC power source is mounted. The power transmission coil, the magnetic sheet, and the power transmission circuit board are disposed in this order from the first wall toward the second wall.

In the power transmission circuit board, a mount surface of the power semiconductor element faces the second wall.

The wireless power transmission device further includes a first TIM material, disposed between the power transmission circuit board and the magnetic sheet, that is in contact with the power transmission circuit board and the magnetic sheet, a second TIM material, disposed between the power semiconductor element and the second wall, that is in contact with the power semiconductor and the second wall, and a heat insulation sheet that is disposed closer to the first wall than is the power transmission coil or a gap between the power transmission coil and the first wall.

The wireless power transmission device forms a first heat path that passes through the first TIM material and has a first average thermal resistance. The wireless power transmission device forms a second heat path that passes through the second TIM material and has a second average thermal resistance. The wireless power transmission device forms a third heat path that includes the heat insulation sheet or the gap and has a third average thermal resistance. The third average thermal resistance is higher than the first average thermal resistance and the second average thermal resistance.

In the structures, the wireless power transmission device thermally conducts heat generated in the power semiconductor element to the magnetic sheet through the first heat path and suppresses local temperature close to the first wall by diffusing the heat in the magnetic sheet. The wireless power transmission device thermally conducts the heat generated in the power semiconductor element to the second wall through the second heat path and suppresses heat generation by radiating the heat from the second wall to an outside of the external housing. The wireless power transmission device suppresses thermal stimulation to a living body by the third heat path insulating the heat reached the first heat path being insulated from the first wall. The wireless power transmission device enhances power efficiency in electromagnetic coupling resonance wireless power feeding by using a magnetic path due to disposition of the power transmission coil and the magnetic sheet and suppresses heat generation due to power loss. The wireless power transmission device suppresses an effect of heat generation on the living body by a temperature control structure including heat conduction, heat diffusion, heat radiation, and heat insulation that use the first heat path, the second heat path, and the third heat path.

According to the present disclosure, the wireless power transmission device according to the present disclosure can suppress the effect of heat generation on the living body and suppress a temperature rise of the living body by using a temperature control structure while achieving high power efficiency in electromagnetic resonance wireless power feeding operation due to electromagnetic coupling that uses an electromagnetic resonance phenomenon.

### BRIEF DESCRIPTION OF THE DRAWINGS

FIG. 1 is a functional block diagram illustrating the schematic structure of a wireless power feeding system according to a first embodiment of the present disclosure;

FIG. 2 is a side cross-sectional view illustrating the structure of a wireless power transmission device according to the first embodiment;

FIG. 3 is an exploded perspective view of the wireless power transmission device according to the first embodiment;

FIG. 4A is a diagram schematically illustrating heat paths, and FIG. 4B is a diagram illustrating an example of heat conduction states of the heat paths;

FIG. 5 is a side cross-sectional view illustrating the structure of a wireless power transmission device according to a second embodiment;

FIG. 6 is a side cross-sectional view illustrating the structure of a wireless power transmission device according to a third embodiment;

FIG. 7 is a side cross-sectional view illustrating the structure of a wireless power transmission device according to a fourth embodiment;

FIG. 8 is a side cross-sectional view illustrating the structure of a wireless power transmission device according to a fifth embodiment; and

FIG. 9 is a side cross-sectional view illustrating the structure of a wireless power transmission device according to a sixth embodiment.

### DETAILED DESCRIPTION

### First Embodiment

A wireless power feeding system that includes a wireless power transmission device according to a first embodiment of the present disclosure will be described with reference to the drawings.

### Functional Structure of Wireless Power Feeding System 1

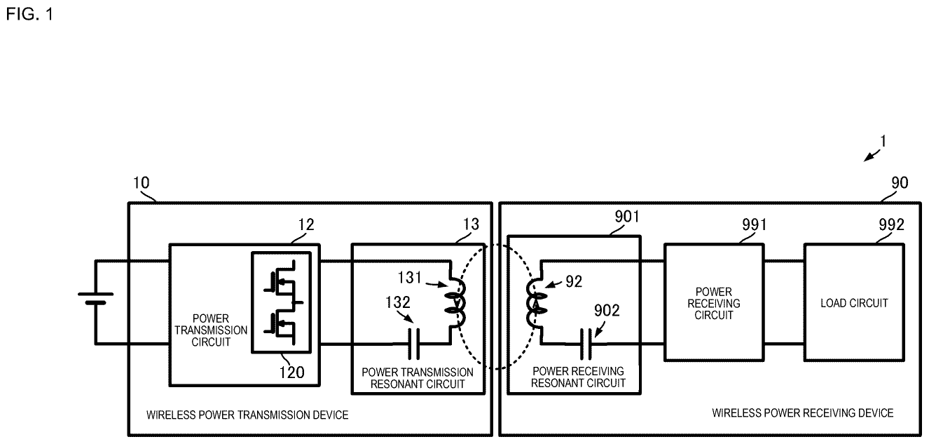

FIG. 1 is a functional block diagram illustrating the schematic structure of a wireless power feeding system according to a first embodiment of the present disclosure. As illustrated in FIG. 1, the wireless power feeding system 1 includes a wireless power transmission device 10 and a wireless power receiving device 90.

Specifically, the wireless power receiving device 90 is an implantable medical device. The wireless power transmission device 10 is disposed outside a living body in which the wireless power receiving device 90 is implanted.

The wireless power transmission device 10 includes a power transmission circuit 12 and a power transmission resonant circuit 13.

The power transmission circuit 12 is connected to a DC power source. The power transmission circuit 12 includes a power semiconductor element 120. The power semiconductor element 120 is a semiconductor that includes a switching element. The power transmission circuit 12 converts DC power from the DC power source into AC power of a predetermined frequency (operating frequency) by switching control of the switching element at the predetermined frequency and generates a power transmission current. The power transmission circuit 12 supplies the power transmission current to the power transmission resonant circuit 13.

The power transmission resonant circuit 13 includes a power transmission coil 131 and a power transmission resonant capacitor 132. The power transmission coil 131 and the power transmission resonant capacitor 132 are connected to each other in series. The resonant frequency (power transmission resonant frequency) of the power transmission resonant circuit 13 is substantially the same as the frequency of the power transmission current.

The power transmission coil 131 generates an alternating magnetic field of the resonant frequency by using the supplied power transmission current.

The wireless power receiving device 90 includes a power receiving resonant circuit 901, a power receiving circuit 991, and a load circuit 992. The load circuit 992 includes, for example, a sensor that obtains various measurement signals for medical use.

The power receiving resonant circuit 901 includes a power receiving coil 92 and a power receiving resonant capacitor 902. The power receiving coil 92 and the power receiving resonant capacitor 902 are connected to each other in series. The resonant frequency (power receiving resonant frequency) of the power receiving resonant circuit 901 is substantially the same as the frequency of the alternating magnetic field, that is, the power transmission resonant frequency.

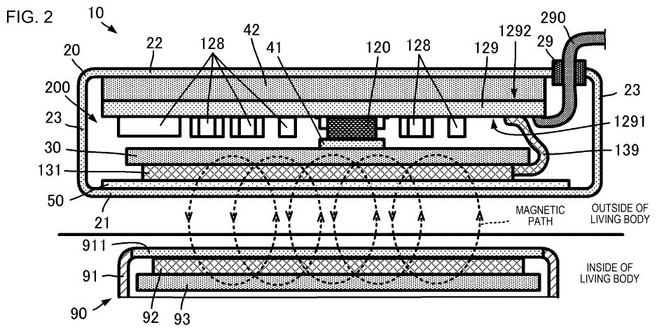

When the power receiving coil 92 is disposed so as to be coupled to the alternating magnetic field (see the magnetic path in FIG. 2 indicated by the dotted line arrows), a power receiving current flows through the power receiving coil 92.

At this time, an electromagnetic resonance field is formed between the power transmission coil 131 and the power receiving coil 92 since the power transmission resonance frequency, the frequency of the alternating magnetic field, and the power receiving resonance frequency are substantially the same as each other.

As a result, the wireless power feeding system 1 can achieve low-loss wireless power feeding from the wireless power transmission device 10 to the wireless power receiving device 90.

It should be noted that the frequency band of the power transmission resonant frequency, the frequency of the alternating magnetic field, and the power receiving resonant frequency, that is, the operating frequency band of the power semiconductor is preferably a 6.78 MHz band or a 13.56 MHz band.

The power receiving resonant circuit 901 outputs the power receiving current to the power receiving circuit 991. The power receiving circuit 991 rectifies and smooths the power receiving current of the power receiving resonance frequency and supplies DC power to the load circuit 992. The load circuit 992 operates by using the supplied DC power.

### Structures of Wireless Power Transmission Device 10 and Wireless Power Receiving Device 90

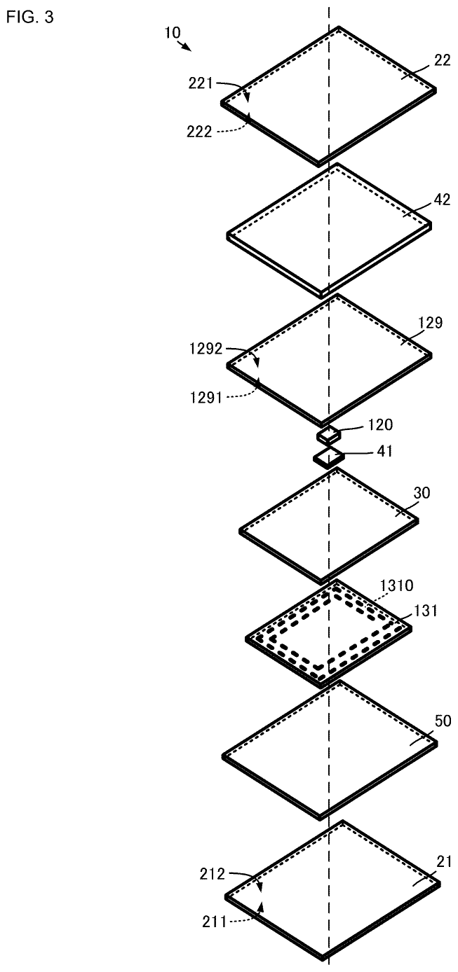

FIG. 2 is a side cross-sectional view illustrating the structure of the wireless power transmission device according to the first embodiment. FIG. 3 is an exploded perspective view of the wireless power transmission device according to the first embodiment. It should be noted that, in FIG. 3, only a portion of the housing is illustrated, and electronic components other than the power semiconductor are not illustrated.

### Schematic Structure of Wireless Power Receiving Device 90

First, before the structure of the wireless power transmission device 10 is described, a portion of the structure of the wireless power receiving device 90 will be described with reference to FIG. 2.

The wireless power receiving device 90 includes a housing 91. The housing 91 has a sealed space therein. The housing 91 is implanted in the living body. The housing 91 incorporates the power receiving coil 92, a power-receiving-side magnetic sheet 93, an electronic circuit board on the power receiving side, and the like. The electronic circuit components that achieve the power receiving resonant capacitor 902, the power receiving circuit 991, and the electronic circuit components that achieve the load circuit 992 are mounted on the electronic circuit board on the power receiving side.

The housing 91 includes a window member 911 disposed on the surface side of the living body. The window member 911 is a flat plate and is disposed substantially parallel to the surface of a portion of the living body in which the wireless power receiving device 90 is implanted.

The power receiving coil 92 has a flat membrane shape and includes an annular conductor that has an axis orthogonal to the flat membrane surface. The power receiving coil 92 is disposed adjacent to the window member 911.

The power receiving side magnetic sheet 93 has a flat membrane shape. The power-receiving-side magnetic sheet 93 is disposed adjacent to the power receiving coil 92 on the opposite side of the power receiving coil 92 from the window member 911.

The electronic circuit board on the power receiving side is disposed on the opposite side of the power-receiving-side magnetic sheet 93 from the power receiving coil 92. In this structure, the magnetic path of the alternating magnetic field is confined by the power-receiving-side magnetic sheet 93 on a wireless power receiving device 90 side (see the dashed arrows in FIG. 2), and the alternating magnetic field is suppressed from leaking to the electronic circuit board on the power receiving side. As a result, the receiving efficiency of the wireless power receiving device 90 is suppressed from being reduced.

The window member 911 is made of a non-metallic biocompatible material. Specifically, the window member 911 is made of sapphire glass, sapphire, ruby, glass, ceramic, or the like. The effect on the living body and the effect from the living body can be suppressed by using such materials as the window member 911. The power receiving coil 92 disposed in the housing 91 can be coupled to the alternating magnetic field generated by the power transmission coil 131.

The portion of the housing 91 other than the window member 911 is made of Ti (titanium), Ti (titanium) alloy (for example, Ti—6Al—4V), or the like. The effect on the living body and the effect from the living body can be suppressed by using such materials as the portion of the housing 91 other than the window member 911. It should be noted that the material mainly containing Ti (titanium) is preferable as a metallic biocompatible material.

In contrast to the wireless power receiving device 90 with the structure described above, the wireless power transmission device 10 has the following structure.

### Structure of Wireless Power Transmission Device 10

As illustrated in FIGS. 2 and 3, the wireless power transmission device 10 includes an external housing 20, a magnetic sheet 30, a first TIM material 41, a second TIM material 42, a heat insulation sheet 50, the power semiconductor element 120, a plurality of electronic circuit components 128, a power transmission circuit board 129, the power transmission coil 131, a wiring pattern 139, and a power source wiring cable 290.

The external housing 20 includes a first wall 21 on a living body facing side, a second wall 22 on a living body opposite side, and a side wall 23. The first wall 21 and the second wall 22 are, for example, rectangular in plan view. It should be noted that the first wall 21 and the second wall 22 are not limited to rectangular. The external housing 20 is made of an insulating resin through which an electromagnetic field passes.

The first wall 21 has an outer surface 211 and an inner surface 212. The second wall 22 has an outer surface 221 and an inner surface 222. The outer surface 211 and the outer surface 221 face the outside of the external housing 20, and the inner surface 212 and the inner surface 222 face the inside (inner space 200 side) of the external housing 20.

The side wall 23 is disposed along the outer periphery of the first wall 21 and the second wall 22 and is connected to the first wall 21 and the second wall 22. As a result, the external housing 20 has an inner space 200. The second wall 22 has a sealing 29, and the power source wiring cable 290 is drawn from the inner space 200 of the external housing 20 through the sealing 29.

The external housing 20 forms the inner space 200, which is a substantially sealed space with this structure. As a result, the external housing 20 can achieve waterproof and dustproof, and the wireless power transmission device 10 has the structure suitable for the power transmission device for medical devices.

The power transmission coil 131 has a flat membrane shape. The power transmission coil 131 includes an annular conductor 1310 that has an axis orthogonal to the flat membrane surface. The power transmission coil 131 is disposed such that the flat membrane surface is parallel to the first wall 21 and the second wall 22 of the external housing 20. The power transmission coil 131 is electrically connected to the power transmission circuit board 129 through the wiring pattern 139.

The power transmission circuit board 129 has a flat plate shape. The power transmission circuit board 129 has a structure in which a conductor pattern is formed on an insulating board. The power transmission circuit board 129 has a mount surface 1291 and a back surface 1292. The power semiconductor element 120 and the plurality of electronic circuit components 128 are mounted on the mount surface 1291.

The magnetic sheet 30 has a flat membrane shape and is made of iron oxide or the like.

The first TIM material 41 and the second TIM material 42 each have a flat membrane shape. The TIM is an abbreviation for a thermal interface material. The first TIM material 41 and the second TIM material 42 are each made of a material with high thermal conductive performance, that is, a material with a high thermal conductivity. The first TIM material 41 and the second TIM material 42 each have a thermal conductivity of, for example, 2.0 W/m·k or higher. For example, the first TIM material 41 and the second TIM material 42 are each made of a material, such as an acrylic-based material, α-gel, Sarcon, or the like.

The heat insulation sheet 50 has a flat membrane shape. The heat insulation sheet 50 is made of a material with high heat insulation performance, that is, a material with low thermal conductivity. The heat insulation sheet 50 has a thermal conductivity of, for example, 0.03 W/m·k or lower.

The power transmission coil 131, the power transmission circuit board 129, the magnetic sheet 30, the first TIM material 41, the second TIM material 42, and the heat insulation sheet 50 are disposed in the inner space 200 of the external housing 20. In this case, the power transmission coil 131, the power transmission circuit board 129, the magnetic sheet 30, the first TIM material 41, the second TIM material 42, and the heat insulation sheet 50 are disposed such that the flat membrane surfaces thereof are parallel to the first wall 21 and the second wall 22.

The power transmission coil 131, the power transmission circuit board 129, the magnetic sheet 30, the first TIM material 41, the second TIM material 42, and the heat insulation sheet 50 are disposed in the order of the heat insulation sheet 50, the power transmission coil 131, the magnetic sheet 30, the first TIM material 41, the power transmission circuit board 129, and the second TIM material 42 from the first wall 21 toward the second wall 22.

More specifically, these components are disposed with respect to the power semiconductor element 120.

First, the disposition from the power transmission circuit board 129 to a first wall 21 side will be described below.

The power transmission circuit board 129 is disposed such that the mount surface 1291 on which the power semiconductor element 120 is mounted faces the first wall 21 side. The first TIM material 41 is disposed on the first wall 21 side of the power semiconductor element 120.

The first TIM material 41 has an area equal to or greater than that of the power semiconductor element 120 in plan view. The first TIM material 41 faces the top surface (the opposite surface from the mount surface) of the power semiconductor element 120. The first TIM material 41 overlaps with substantially the entire surface of the power semiconductor element 120 and is in contact with the power semiconductor element 120.

The magnetic sheet 30 has an area that is greater than that of the first TIM material 41 and is equal to or greater than that of the power transmission coil 131 in plan view. The magnetic sheet 30 faces the mount surface 1291 of the power transmission circuit board 129 and the first TIM material 41. The magnetic sheet 30 overlaps with substantially the entire surface of the first TIM material 41 and is in contact with the first TIM material 41.

The power transmission coil 131 faces the magnetic sheet 30. The power transmission coil 131 overlaps with the magnetic sheet 30 and is in contact with the magnetic sheet 30.

The heat insulation sheet 50 has an area equal to or greater than that of the power transmission coil 131 in plan view. The heat insulation sheet 50 faces the power transmission coil 131. The heat insulation sheet 50 overlaps with substantially the entire surface of the power transmission coil 131 and is in contact with the power transmission coil 131.

The heat insulation sheet 50 faces the inner surface 212 of the first wall 21 and is in contact with the inner surface 212. It should be noted that the heat insulation sheet 50 does not need to be in contact with the inner surface 212 of the first wall 21. However, when the heat insulation sheet 50 is in contact with the inner surface 212 of the first wall 21, the distance between the power transmission coil 131 and the first wall 21 can be reduced.

Next, the disposition from the power transmission circuit board 129 to a second wall 22 side will be described below.

The second TIM material 42 has an area equal to or greater than that of the power transmission circuit board 129 in plan view. The second TIM material 42 overlaps with substantially the entire surface of the power transmission circuit board 129 in plan view and is in contact with the power transmission circuit board 129.

The second TIM material 42 is in contact with the inner surface 222 of the second wall 22.

In the structure described above, when the power semiconductor element 120 is driven and generates heat, the heat from the power semiconductor element 120, which is the heat source, is conducted as described below.

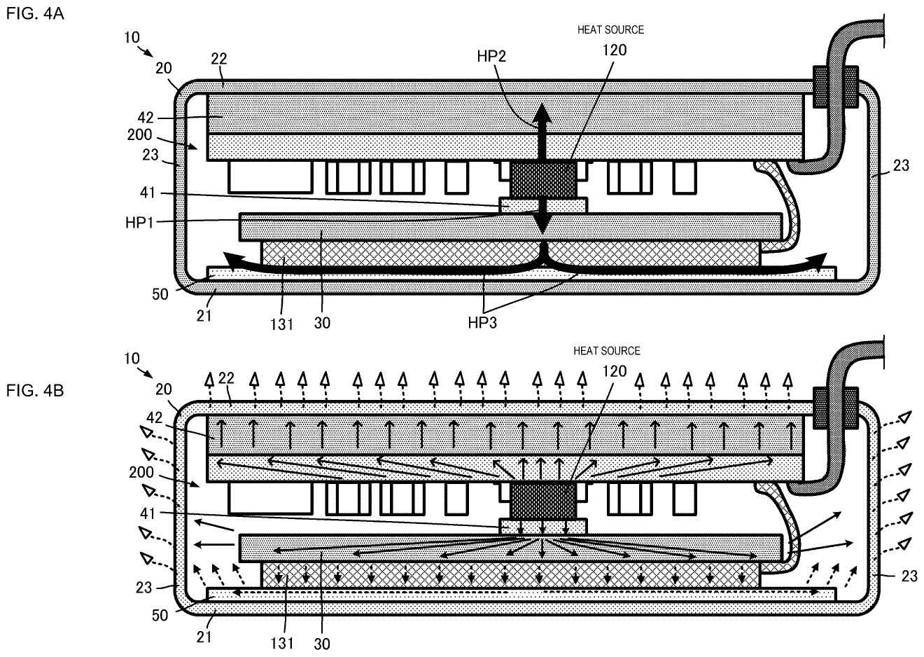

FIG. 4A is a diagram schematically illustrating heat paths, and FIG. 4B is a diagram illustrating an example of heat conduction states of the heat paths.

As illustrated in FIGS. 4A and 4B, the heat generated by the power semiconductor element 120 is conducted to the first wall 21 side and the second wall 22 side.

### First Wall 21 Side

On the first wall 21 side, the heat of the power semiconductor element 120 is conducted to the first TIM material 41. The heat that has been conducted to the first TIM material 41 is conducted to the magnetic sheet 30. The magnetic sheet 30 has an area greater than that of the first TIM material 41. That is, the heat that has been conducted to the first TIM material 41 diffuses into the magnetic sheet 30.

The heat conduction path including the first TIM material 41 and the magnetic sheet 30 is a first heat path HP1.

The heat that has been conducted to the surface of the magnetic sheet 30 that is in contact with the power transmission coil 131 is conducted to the power transmission coil 131. It should be noted that the heat that has been conducted to a portion of the magnetic sheet 30 excluding the contact surface with the power transmission coil 131 diffuses, for example, from the magnetic sheet 30 into the inner space 200.

The heat that has been conducted to the power transmission coil 131 reaches the surface of the heat insulation sheet 50.

The heat conduction path including the power transmission coil 131 and the heat insulation sheet 50 is a third heat path HP3.

### Second Wall 22 Side

On the second wall 22 side, the heat of the power semiconductor element 120 is conducted to the second TIM material 42 through the power transmission circuit board 129. The heat that has been conducted to the second TIM material 42 is conducted to the second wall 22.

The heat conduction path including the power transmission circuit board 129 and the second TIM material 42 is a second heat path HP2.

In this structure, the first heat path HP1 includes the first TIM material 41, the second heat path HP2 includes the second TIM material 42, and the third heat path HP3 includes the heat insulation sheet 50. Accordingly, the first average thermal resistance of the first heat path HP1 and the second average thermal resistance of the second heat path HP2 are lower than the third average thermal resistance of the third heat path HP3.

As a result, for the second wall 22 side, the heat of the power semiconductor element 120 is effectively conducted to the second wall 22 of the external housing 20 through the second heat path HP2. The heat that has been conducted to the second wall 22 is radiated to the outside of the external housing 20 mainly from the second wall 22.

On the other hand, for the first wall 21 side, the heat of the power semiconductor element 120 is effectively conducted to the power transmission coil 131 through the first heat path HP1. However, the third heat path HP3 suppresses the heat from being conducted to the first wall 21. As a result, a temperature rise of the first wall 21 is suppressed.

It should be noted that the heat that has been diffused through the first heat path HP1 and the heat insulated by the third heat path HP3 are diffused into the inner space 200. The heat that has been conducted to the inner space 200 is radiated to the outside of the external housing 20 through the side wall 23 and the second wall 22 of the external housing 20.

In this case, the thermal conductivity of the external housing 20 is preferably set to lower than the thermal conductivities of the first TIM material 41 and the second TIM material 42 and higher than the thermal conductivity of the heat insulation sheet 50. As a result, the heat can be suppressed from being conducted to the first wall 21 through the side wall 23 while heat radiation through the second wall 22 and the side wall 23 is achieved, and accordingly, a temperature rise of the first wall 21 can be suppressed.

As described above, the wireless power transmission device 10 conducts the heat generated in the power semiconductor element 120 to the magnetic sheet 30 through the first heat path HP1 and suppresses the local temperature close to the first wall 21 due to heat diffusion in the magnetic sheet 30.

The wireless power transmission device 10 conducts the heat generated in the power semiconductor element 120 to the second wall 22 through the second heat path HP2 and suppresses heat generation by radiating the heat from the second wall 22 to the outside of the external housing 20.

The wireless power transmission device 10 suppresses thermal stimulation to the living body by the third heat path HP3 insulating the heat reached the first heat path HP1 from the first wall 21.

In addition, the wireless power transmission device 10 forms, with greater certainty, an electromagnetic coupling resonance field (see the dotted arrows in FIG. 2) due to electromagnetic coupling that uses an electromagnetic resonance phenomenon by using a magnetic path due to the disposition of the power transmission coil 131 and the magnetic sheet 30. As a result, the wireless power transmission device 10 enhances the power efficiency in electromagnetic resonance wireless power feeding operation. Accordingly, the wireless power transmission device 10 suppresses heat generation due to power loss.

As described above, the wireless power transmission device 10 can suppress the effect of heat generation on the living body and can achieve high power feeding efficiency by temperature control, such as heat conduction, heat diffusion, heat radiation, and heat insulation by using the first heat path HP1, the second heat path HP2, and the third heat path HP3.

### Second Embodiment

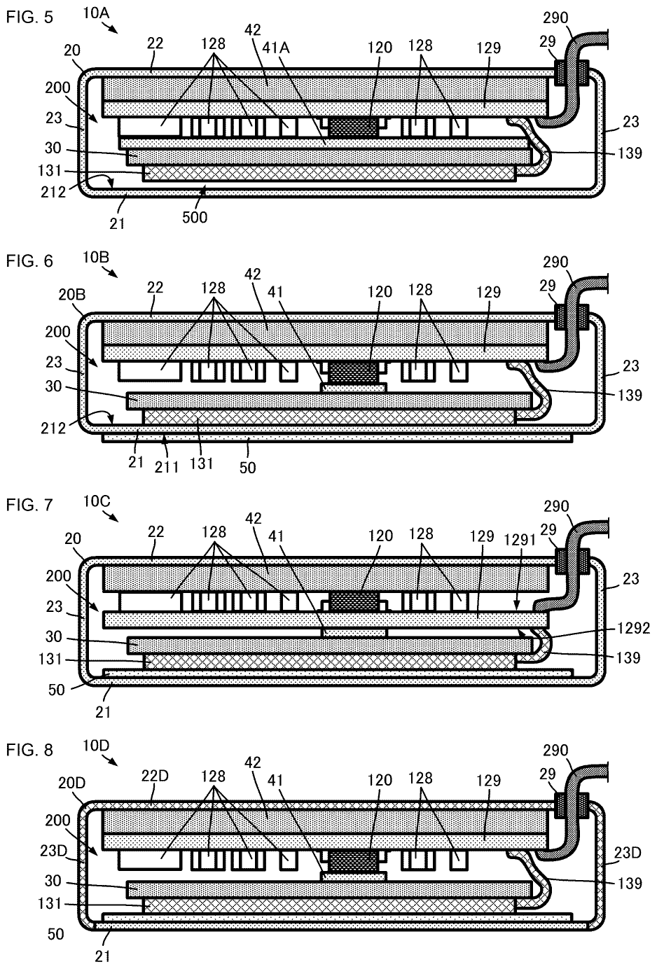

A wireless power transmission device according to a second embodiment of the present disclosure will be described with reference to the drawings. FIG. 5 is a side cross-sectional view illustrating the structure of the wireless power transmission device according to the second embodiment.

As illustrated in FIG. 5, a wireless power transmission device 10A according to the second embodiment differs from the wireless power transmission device 10 according to the first embodiment in that the wireless power transmission device 10A includes a first TIM material 41A and a gap 500. The remaining structure of the wireless power transmission device 10A according to the second embodiment is similar to that of the wireless power transmission device 10 according to the first embodiment, and the description of the similar structure is omitted.

In the wireless power transmission device 10A, the first TIM material 41A has an area substantially the same as that of the power transmission circuit board 129. The first TIM material 41A is disposed entirely on the surface of the power transmission circuit board 129 with which the magnetic sheet 30 overlaps.

In this case, the first TIM material 41A is preferably in contact with electronic circuit components other than the power semiconductor element 120 that are mounted on the power transmission circuit board 129 and generate heat. As a result, the wireless power transmission device 10A can improve the overall heat radiation effect of the power transmission circuit board 129 through the first heat path HP1.

The wireless power transmission device 10A includes the gap 500 between the power transmission coil 131 and the inner surface 212 of the first wall 21. Since the gap 500 is an air layer of a predetermined thickness, the thermal conductivity is low. Accordingly, the gap 500 can achieve a heat insulation function similar to that of the heat insulation sheet 50.

With the structure described above, the wireless power transmission device 10A exhibits the same operation and effect as the wireless power transmission device 10.

It should be noted that the wireless power transmission device 10A may also have either the structure that includes the large-area first TIM material 41A or the structure that includes the gap 500. For example, the wireless power transmission device 10A may have the structure that includes the gap 500 and the first TIM material 41 according to the first embodiment or the structure that includes the first TIM material 41A and the heat insulation sheet 50 according to the first embodiment.

### Third Embodiment

A wireless power transmission device according to a third embodiment of the present disclosure will be described with reference to the drawings. FIG. 6 is a side cross-sectional view illustrating the structure of the wireless power transmission device according to the third embodiment.

As illustrated in FIG. 6, a wireless power transmission device 10B according to the third embodiment differs from the wireless power transmission device 10 according to the first embodiment in the disposition of the heat insulation sheet 50. The remaining structure of the wireless power transmission device 10B according to the third embodiment is similar to that of the wireless power transmission device 10 according to the first embodiment, and the description of the similar structure is omitted.

The heat insulation sheet 50 is disposed so as to be in contact with the outer surface 211 of the first wall 21 of an external housing 20B. The heat insulation sheet 50 is disposed so as to overlap with the entire surface of the power transmission coil 131 in plan view and is disposed on substantially the entire outer surface 211 of the first wall 21.

The power transmission coil 131 is in contact with the inner surface 212 of the first wall 21.

With the structure described above, the wireless power transmission device 10B exhibits the same operation and effect as the wireless power transmission device 10. In addition, in the wireless power transmission device 10B, even when the wireless power transmission device 10B is in contact with the surface of the living body, the heat insulation sheet 50 is in direct contact with the surface. As a result, the wireless power transmission device 10B can suppress thermal stimulation to the living body with greater certainty.

It should be noted that the wireless power transmission device 10B may have a gap between the power transmission coil 131 and the inner surface 212 of the first wall 21. As a result, the thermal insulation effect on the living body is further improved. In this case, the thickness of the gap is preferably smaller (for example, smaller than the thickness of the heat insulation sheet 50). As a result, the distance between the power transmission coil 131 and the power receiving coil 92 can be minimized while the thermal insulation effect is improved.

### Fourth Embodiment

A wireless power transmission device according to a fourth embodiment of the present disclosure will be described with reference to the drawings. FIG. 7 is a side cross-sectional view illustrating the structure of the wireless power transmission device according to the fourth embodiment.

As illustrated in FIG. 7, a wireless power transmission device 10C according to the fourth embodiment differs from the wireless power transmission device 10 according to the first embodiment in that the mount surface 1291 and the back surface 1292 of the power transmission circuit board 129 are reversed from each other. The remaining structure of the wireless power transmission device 10C according to the fourth embodiment is similar to that of the wireless power transmission device 10 according to the first embodiment, and the description of the similar structure is omitted.

The power transmission circuit board 129 is disposed such that the mount surface 1291 faces the second wall 22. In other words, the back surface 1292 of the power transmission circuit board 129 faces the first wall 21.

The first TIM material 41 is in contact with the back surface 1292 of the power transmission circuit board 129. In this case, the first TIM material 41 is disposed so as to overlap with the power semiconductor element 120 in plan view.

It should be noted that the first TIM material 41A as illustrated in the second embodiment may be used instead of the first TIM material 41. In this case, the first TIM material 41A is in contact with substantially the entire back surface 1292.

The second TIM material 42 is in contact with the power semiconductor element 120.

With the structure described above, the wireless power transmission device 10C exhibits the same operation and effect as the wireless power transmission device 10. In addition, in the wireless power transmission device 10C, the power transmission circuit board 129 inserted between the power semiconductor element 120, which is a heat source, and the first wall 21 (on the living body side). As a result, the wireless power transmission device 10C can further suppress heat conduction to the first wall 21 side.

### Fifth Embodiment

A wireless power transmission device according to a fifth embodiment of the present disclosure will be described with reference to the drawings. FIG. 8 is a side cross-sectional view illustrating the structure of the wireless power transmission device according to the fifth embodiment.

As illustrated in FIG. 8, a wireless power transmission device 10D according to the fifth embodiment differs from the wireless power transmission device 10 according to the first embodiment in that the wireless power transmission device 10D includes an external housing 20D. The remaining structure of the wireless power transmission device 10D according to the fifth embodiment is similar to that of the wireless power transmission device 10 according to the first embodiment, and the description of the similar structure is omitted.

The external housing 20D includes the first wall 21, a second wall 22D, and a side wall 23D. The first wall 21 is made of an insulating resin through which an electromagnetic field passes. The second wall 22D and the side wall 23D are each made of a metal.

With the structure described above, the wireless power transmission device 10D exhibits the same operation and effect as the wireless power transmission device 10. In addition, in the wireless power transmission device 10D, the second wall 22D and the side wall 23D are each made of a metal and have a high thermal conductivity. Accordingly, the wireless power transmission device 10D can achieve a higher heat radiation effect while achieving wireless power feeding.

It should be noted that the second wall 22D and the side wall 23D of the wireless power transmission device 10D are each made of a metal, but the second wall 22D may be made of a metal, and the side wall 23D may be made of an insulating resin. In addition, the portion of the side wall 23D on the second wall 22D side may be made of a metal, and the portion of the side wall 23D on the first wall 21 side may be made of an insulating resin.

### Sixth Embodiment

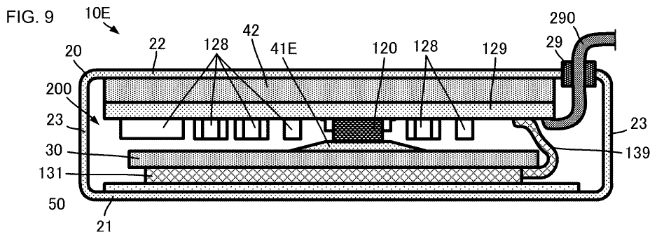

A wireless power transmission device according to a sixth embodiment of the present disclosure will be described with reference to the drawings. FIG. 9 is a side cross-sectional view illustrating the structure of the wireless power transmission device according to the sixth embodiment.

As illustrated in FIG. 9, a wireless power transmission device 10E according to the sixth embodiment differs from the wireless power transmission device 10 according to the first embodiment in that the wireless power transmission device 10E includes a first TIM material 41E. The remaining structure of the wireless power transmission device 10E according to the sixth embodiment is similar to that of the wireless power transmission device 10 according to the first embodiment, and the description of the similar structure is omitted.

The first TIM material 41E has a three-dimensional shape in which the area of the surface in contact with the magnetic sheet 30 is greater than the area of the surface in contact with the power semiconductor element 120.

With the structure described above, the wireless power transmission device 10E exhibits the same operation and effect as the wireless power transmission device 10. In addition, the wireless power transmission device 10E can more easily achieve heat diffusion in the magnetic sheet 30 during heat conduction from the power semiconductor element 120 to the magnetic sheet 30.

The structures of the embodiments described above can be combined as appropriate, and the operation and effect corresponding to each of the combinations can be achieved.
<1> A wireless power transmission device comprising an external housing including a first wall on a living body facing side and a second wall on a living body opposite side; a power transmission coil disposed in the external housing; a power transmission resonant capacitor that constitutes a resonant circuit together with the power transmission coil; a magnetic sheet disposed in the external housing; and a power transmission circuit board on which a power semiconductor element handling a DC power source is mounted, the power transmission circuit board being disposed in the external housing. The power transmission coil, the magnetic sheet, and the power transmission circuit board are disposed in this order from the first wall toward the second wall. In the power transmission circuit board, a mount surface of the power semiconductor element faces the magnetic sheet. The wireless power transmission device further comprises a first TIM material, disposed between the power semiconductor element and the magnetic sheet, that is in contact with the power semiconductor element and the magnetic sheet; a second TIM material, disposed between the power transmission circuit board and the second wall, that is in contact with the power transmission circuit board and the second wall; and a heat insulation sheet that is disposed closer to the first wall than is the power transmission coil or a gap between the power transmission coil and the first wall. A first heat path that passes through the first TIM material and has a first average thermal resistance is formed. A second heat path that passes through the second TIM material and has a second average thermal resistance is formed. A third heat path that includes the heat insulation sheet or the gap and has a third average thermal resistance is formed. The third average thermal resistance is higher than the first average thermal resistance and the second average thermal resistance. Heat generated in the power semiconductor element is thermally conducted to the magnetic sheet through the first heat path, and local temperature close to the first wall is suppressed by the heat being diffused in the magnetic sheet. The heat generated in the power semiconductor element is thermally conducted to the second wall through the second heat path, and heat generation is suppressed by the heat being radiated from the second wall to an outside of the external housing. Thermal stimulation to a living body is suppressed by the third heat path insulating the heat reached the first heat path from the first wall. Power efficiency in electromagnetic resonance wireless power feeding operation due to electromagnetic coupling that uses electromagnetic resonance phenomenon is enhanced by using a magnetic path due to disposition of the power transmission coil and the magnetic sheet, and heat generation due to power loss is suppressed. Also, an effect of heat generation on the living body is suppressed by a temperature control structure including heat conduction, heat diffusion, heat radiation, and heat insulation that use the first heat path, the second heat path, and the third heat path.
<2> A wireless power transmission device comprising an external housing including a first wall on a living body facing side and a second wall on a living body opposite side; a power transmission coil disposed in the external housing; a power transmission resonant capacitor that constitutes a resonant circuit together with the power transmission coil; a magnetic sheet disposed in the external housing; and a power transmission circuit board on which a power semiconductor element handling a DC power source is mounted, the power transmission circuit board being disposed in the external housing. The power transmission coil, the magnetic sheet, and the power transmission circuit board are disposed in this order from the first wall toward the second wall. In the power transmission circuit board, a mount surface of the power semiconductor element faces the second wall. The wireless power transmission device further comprises a first TIM material, disposed between the power transmission circuit board and the magnetic sheet, that is in contact with the power transmission circuit board and the magnetic sheet; a second TIM material, disposed between the power semiconductor element and the second wall, that is in contact with the power semiconductor element and the second wall; and a heat insulation sheet that is disposed closer to the first wall than is the power transmission coil or a gap between the power transmission coil and the first wall. A first heat path that passes through the first TIM material and has a first average thermal resistance is formed. A second heat path that passes through the second TIM material and has a second average thermal resistance is formed. A third heat path that includes the heat insulation sheet or the gap and has a third average thermal resistance is formed. The third average thermal resistance is higher than the first average thermal resistance and the second average thermal resistance. Heat generated in the power semiconductor element is thermally conducted to the magnetic sheet through the first heat path, and local temperature close to the first wall is suppressed by the heat being diffused in the magnetic sheet. The heat generated in the power semiconductor element is thermally conducted to the second wall through the second heat path, and heat generation is suppressed by the heat being radiated from the second wall to an outside of the external housing. Thermal stimulation to a living body is suppressed by the third heat path insulating the heat reached the first heat path from the first wall. Power efficiency in electromagnetic resonance wireless power feeding operation due to electromagnetic coupling that uses electromagnetic resonance phenomenon is enhanced by using a magnetic path due to disposition of the power transmission coil and the magnetic sheet, and heat generation due to power loss is suppressed. Also, an effect of heat generation on the living body is suppressed by a temperature control structure including heat conduction, heat diffusion, heat radiation, and heat insulation that use the first heat path, the second heat path, and the third heat path.
<3> The wireless power transmission device according to <1> or <2>, wherein a thermal conductivity of the first TIM material and a thermal conductivity of the second TIM material are 2.0 W/m·k or higher.
<4> The wireless power transmission device according to any one of <1> to <3>, wherein a thermal conductivity of the heat insulation sheet is 0.03 W/m·k or lower.
<5> The wireless power transmission device according to any one of <1> to <4>, wherein a thermal conductivity of the first wall is lower than a thermal conductivity of the second wall.
<6> The wireless power transmission device according to <5>, wherein the first wall includes an insulating resin, and the second wall includes a metal.
<7> The wireless power transmission device according to any one of <1> to <6>, wherein an operating frequency band of the power semiconductor element is a 6.78 MHz band or a 13.56 MHz band.
<8> The wireless power transmission device according to any one of <1> to <7>, wherein the first TIM material is disposed entirely on a surface of the power transmission circuit board close to the magnetic sheet with which the magnetic sheet overlaps.
<9> The wireless power transmission device according to any one of <1> to <8>, wherein the second TIM material is disposed entirely on a surface of the power transmission circuit board close to the second wall.
<10> The wireless power transmission device according to any one of <1> to <9>, wherein, when the heat insulation sheet is used, the heat insulation sheet is disposed in the external housing.
<11> The wireless power transmission device according to any one of <1> to <10>, wherein, when the heat insulation sheet is used, the heat insulation sheet is disposed on an outer surface of the first wall.

What is claimed is:

1. A wireless power transmission device comprising:
an external housing including a first wall on a living body facing side and a second wall on a living body opposite side;
a power transmission coil in the external housing;
a power transmission resonant capacitor that configures a resonant circuit together with the power transmission coil;
a magnetic sheet in the external housing; and
a power transmission circuit board on which a power semiconductor element handling a DC power source is mounted, the power transmission circuit board being in the external housing,
wherein
the power transmission coil, the magnetic sheet, and the power transmission circuit board are disposed in this order from the first wall toward the second wall,
in the power transmission circuit board, a mount surface of the power semiconductor element faces the magnetic sheet,
the wireless power transmission device further comprising:
a first TIM material, between the power semiconductor element and the magnetic sheet, that is in contact with the power semiconductor element and the magnetic sheet;
a second TIM material, between the power transmission circuit board and the second wall, that is in contact with the power transmission circuit board and the second wall; and
a heat insulation sheet that is closer to the first wall than is the power transmission coil or a gap between the power transmission coil and the first wall,
wherein
a first heat path passes through the first TIM material and has a first average thermal resistance,
a second heat path passes through the second TIM material and has a second average thermal resistance,
a third heat path includes the heat insulation sheet or the gap and has a third average thermal resistance,
the third average thermal resistance is higher than the first average thermal resistance and the second average thermal resistance,
heat generated in the power semiconductor element is thermally conducted to the magnetic sheet through the first heat path, and local temperature close to the first wall is suppressed by the heat being diffused in the magnetic sheet,
the heat generated in the power semiconductor element is thermally conducted to the second wall through the second heat path, and heat generation is suppressed by the heat being radiated from the second wall to an outside of the external housing,
thermal stimulation to a living body is suppressed by the third heat path insulating the heat reached the first heat path from the first wall,
power efficiency in electromagnetic resonance wireless power feeding operation due to electromagnetic coupling that uses electromagnetic resonance phenomenon is enhanced by using a magnetic path due to disposition of the power transmission coil and the magnetic sheet, and heat generation due to power loss is suppressed, and
an effect of heat generation on the living body is suppressed by a temperature control structure including heat conduction, heat diffusion, heat radiation, and heat insulation that use the first heat path, the second heat path, and the third heat path.

2. A wireless power transmission device comprising:
an external housing including a first wall on a living body facing side and a second wall on a living body opposite side;
a power transmission coil in the external housing;
a power transmission resonant capacitor that configures a resonant circuit together with the power transmission coil;
a magnetic sheet in the external housing; and
a power transmission circuit board on which a power semiconductor element handling a DC power source is mounted, the power transmission circuit board being in the external housing,
wherein
the power transmission coil, the magnetic sheet, and the power transmission circuit board are in this order from the first wall toward the second wall,
in the power transmission circuit board, a mount surface of the power semiconductor element faces the second wall,
the wireless power transmission device further comprises:
a first TIM material, between the power transmission circuit board and the magnetic sheet, that is in contact with the power transmission circuit board and the magnetic sheet;
a second TIM material, between the power semiconductor element and the second wall, that is in contact with the power semiconductor element and the second wall; and
a heat insulation sheet that is closer to the first wall than is the power transmission coil or a gap between the power transmission coil and the first wall,
a first heat path that passes through the first TIM material and has a first average thermal resistance is formed,
a second heat path that passes through the second TIM material and has a second average thermal resistance is formed,
a third heat path that includes the heat insulation sheet or the gap and has a third average thermal resistance is formed,
the third average thermal resistance is higher than the first average thermal resistance and the second average thermal resistance,
heat generated in the power semiconductor element is thermally conducted to the magnetic sheet through the first heat path, and local temperature close to the first wall is suppressed by the heat being diffused in the magnetic sheet,
the heat generated in the power semiconductor element is thermally conducted to the second wall through the second heat path, and heat generation is suppressed by the heat being radiated from the second wall to an outside of the external housing,
thermal stimulation to a living body is suppressed by the third heat path insulating the heat reached the first heat path from the first wall,
power efficiency in electromagnetic resonance wireless power feeding operation due to electromagnetic coupling that uses electromagnetic resonance phenomenon is enhanced by using a magnetic path due to disposition of the power transmission coil and the magnetic sheet, and heat generation due to power loss is suppressed, and
an effect of heat generation on the living body is suppressed by a temperature control structure including heat conduction, heat diffusion, heat radiation, and heat insulation that use the first heat path, the second heat path, and the third heat path.

3. The wireless power transmission device according to claim 1, wherein
a thermal conductivity of the first TIM material and a thermal conductivity of the second TIM material are 2.0 W/m·k or higher.

4. The wireless power transmission device according to claim 1, wherein
a thermal conductivity of the heat insulation sheet is 0.03 W/m·k or lower.

5. The wireless power transmission device according to claim 1, wherein
a thermal conductivity of the first wall is lower than a thermal conductivity of the second wall.

6. The wireless power transmission device according to claim 5, wherein
the first wall includes an insulating resin, and
the second wall includes a metal.

7. The wireless power transmission device according to claim 1, wherein
an operating frequency band of the power semiconductor element is a 6.78 MHz band or a 13.56 MHz band.

8. The wireless power transmission device according to claim 1, wherein
the first TIM material is entirely on a surface of the power transmission circuit board close to the magnetic sheet with which the magnetic sheet overlaps.

9. The wireless power transmission device according to claim 1, wherein
the second TIM material is entirely on a surface of the power transmission circuit board close to the second wall.

10. The wireless power transmission device according to claim 1, wherein
when the heat insulation sheet is used, the heat insulation sheet is in the external housing.

11. The wireless power transmission device according to claim 1, wherein
when the heat insulation sheet is used, the heat insulation sheet is on an outer surface of the first wall.

12. The wireless power transmission device according to claim 2, wherein
a thermal conductivity of the first TIM material and a thermal conductivity of the second TIM material are 2.0 W/m·k or higher.

13. The wireless power transmission device according to claim 2, wherein
a thermal conductivity of the heat insulation sheet is 0.03 W/m·k or lower.

14. The wireless power transmission device according to claim 2, wherein
a thermal conductivity of the first wall is lower than a thermal conductivity of the second wall.

15. The wireless power transmission device according to claim 14, wherein
the first wall includes an insulating resin, and
the second wall includes a metal.

16. The wireless power transmission device according to claim 2, wherein
an operating frequency band of the power semiconductor element is a 6.78 MHz band or a 13.56 MHz band.

17. The wireless power transmission device according to claim 2, wherein
the first TIM material is entirely on a surface of the power transmission circuit board close to the magnetic sheet with which the magnetic sheet overlaps.

18. The wireless power transmission device according to claim 2, wherein
the second TIM material is entirely on a surface of the power transmission circuit board close to the second wall.

19. The wireless power transmission device according to claim 2, wherein
when the heat insulation sheet is used, the heat insulation sheet is in the external housing.

20. The wireless power transmission device according to claim 2, wherein
when the heat insulation sheet is used, the heat insulation sheet is on an outer surface of the first wall.
